(19) United States
(12) Patent Application Publication
(10) Pub. No.: US 2024/0420388 A1
(43) Pub. Date: Dec. 19, 2024

(54) CONE BEAM COMPUTED TOMOGRAPHY RECONSTRUCTION

(71) Applicant: Elekta Limited, Crawley (GB)

(72) Inventors: Rui Lopes, Brossard (CA); Samuele PAPA, Crawley (GB); David KNIGGE, Crawley (GB); Efstratios GAVVES, Crawley (GB); Jan-Jakob Sonke, Amsterdam (NL)

(21) Appl. No.: 18/744,831

(22) Filed: Jun. 17, 2024

(30) Foreign Application Priority Data
Jun. 16, 2023 (GB) 2309028.5

(51) Int. Cl. G06T 11/00; A61B 6/03; A61B 6/40

(52) U.S. Cl. CPC G06T 11/006; A61B 6/032; A61B 6/4085; G06T 2211/441

(57) ABSTRACT

A computer implemented method for reconstructing a volumetric medical image of a patient from Cone Beam Computed Tomography (CBCT) projections of the patient can comprise using a shared Neural Field (NF) to generate a volumetric field of attenuation coefficients from the CBCT projections. The shared NF can be modulated by a patient specific NF. The method can further comprise mapping the volumetric field of attenuation coefficients to a volumetric image of the patient.

### CLAIM FOR PRIORITY

This application claims the benefit of priority of British Application Serial No. 2309028.5, filed Jun. 16, 2023, which is hereby incorporated by reference in its entirety.

### TECHNICAL FIELD

The present disclosure relates to a method for reconstructing a volumetric medical image of a patient from Cone Beam Computed Tomography (CBCT) projections of the patient. The present disclosure also relates to a method for training a shared Neural Field for use in reconstructing a volumetric medical image of a patient from CBCT projections of the patient. The present disclosure also relates to a reconstruction node, a training node, and to a computer program product configured, when run on a computer, to carry out methods for reconstructing a volumetric image of a patient and for training a shared Neural Field.

### BACKGROUND

In inverse problems, the goal is to infer a certain quantity of interest from indirect observations. This type of problem arises in many scientific fields including medical imaging, biology, and physics. Unfortunately, many inverse problems are inherently ill-posed, i.e., there exist multiple solutions that agree with the measurements, and these do not necessarily depend continuously on the data. Tools from machine learning, and deep learning in particular, have attracted a lot of attention in research into these issues.

Computed Tomography (CT) is a medical imaging technique for reconstructing material density inside a patient, using the mathematical and physical properties of X-ray scanners. To be precise, CT aims to produce attenuation coefficients of patient tissue, as they are strongly related to density under assumptions that hold in the CT setting. In CT, several X-ray scans, or projections, of the patient are acquired from various angles using a detector. An important variant of CT is Cone Beam CT (CBCT), which uses flat panel detectors to scan a large fraction of the volume in a single rotation. CBCT reconstruction is more difficult than reconstruction for classical (helical) CT, owing to the inherent mathematical difficulty of Radon Transform inversion in the three-dimensional setting, physical limits of the detector, and characteristics of the measurement process such as noise. Traditional reconstruction methods used for CBCT include FDK (Feldkamp et al., 1984), and iterative reconstruction, such as that disclosed in Kaipio & Somersalo, 2005. FDK filters the projections and applies other simple corrections to account for the physical geometry of the acquisition system. Iterative methods use optimization to find the density that most closely resembles the measurements once projected using a forward operator. In addition, deep learning has seen increasing use in the field, with algorithms such as learned primal-dual (Adler & Oktem, 2018), invertible learned primal-dual (Rudzusika et al., 2021) and LIRE (Moriakov et al., 2022).

### SUMMARY

An important use for CBCT is in the planning and delivery of Radiotherapy, which may be used to treat cancers or other conditions in human or animal tissue. The treatment planning procedure for radiotherapy may include using a three dimensional image of the patient to identify a target region, for example the tumour, and to identify organs near the tumour, termed Organs at Risk (OARs). A treatment plan aims to ensure delivery of a required dose of radiation to the tumour, while minimising the risk to nearby OARs. A treatment plan for a patient may be generated in an offline manner, using medical images that have been obtained using, for example classical CT. These images are generally referred to in this context as diagnostic or planning CT images. The radiation treatment plan includes parameters specifying the direction, cross sectional shape, and intensity of each radiation beam to be applied to the patient. The radiation treatment plan may include dose fractioning, in which a sequence of radiation treatments is provided over a predetermined period of time, with each treatment delivering a specified fraction of the total prescribed dose. Multiple patient images may be required during the course of radiotherapy treatment, and owing to their speed, convenience, and lower cost, CBCT images, as opposed to classical CT images, may be used to determine changes in patient anatomy between delivery of individual dose fractions.

This disclosure describes, among other things, a method, a reconstruction node, a training node, and a computer program product which at least partially address one or more of the challenges mentioned above. This disclosure also describes, among other things a method, a reconstruction node, a training node, and a computer program product which cooperate to perform reconstruction of a volumetric medical image of a patient from CBCT projections of the patient, which method achieves improved performance when compared with existing methods.

According to a first aspect of the present disclosure, there is provided a computer implemented method for reconstructing a volumetric medical image of a patient from CBCT projections of the patient. The method comprises using a shared Neural Field (NF) to generate a volumetric field of attenuation coefficients from the CBCT projections, wherein the shared NF is modulated by a patient specific NF, and mapping the volumetric field of attenuation coefficients to a volumetric image of the patient.

According to another aspect of the present disclosure, there is provided a computer implemented method for training a shared NF for use in reconstructing a volumetric medical image of a patient from CBCT projections of the patient, wherein the shared NF is operable to generate a volumetric field of attenuation coefficients from the CBCT projections, and wherein the shared NF is modulated by a patient specific NF. The method comprises training the shared NF by using as ground truth volumetric medial images reconstructed from diagnostic CT projections of patients other than the patient for which the shared NF will be used.

According to another aspect of the present disclosure, there is provided a computer program product comprising a computer readable medium, the computer readable medium having computer readable code embodied therein, the computer readable code being configured such that, on execution by a suitable computer or processor, the computer or processor is caused to perform a method according to any one or more aspects or examples of the present disclosure.

According to another aspect of the present disclosure, there is provided a reconstruction node for reconstructing a volumetric medical image of a patient from CBCT projections of the patient. The reconstruction node comprises processing circuitry configured to cause the reconstruction node to use a shared NF to generate a volumetric field of attenuation coefficients from the CBCT projections, wherein the shared NF is modulated by a patient specific NF, and to map the volumetric field of attenuation coefficients to a volumetric image of the patient.

According to another aspect of the present disclosure, there is provided a training node for training a shared Neural Field for use in reconstructing a volumetric medical image of a patient from CBCT projections of the patient, wherein the shared NF is operable to generate a volumetric field of attenuation coefficients from the CBCT projections, and wherein the shared NF is modulated by a patient specific NF. The training node comprises processing circuitry configured to cause the training node to train the shared NF by using as ground truth volumetric medial images reconstructed from diagnostic CT projections of patients other than the patient for which the shared NF will be used.

According to another aspect of the present disclosure, there is provided radiotherapy treatment apparatus comprising at least one of a reconstruction node and/or a training node according to any one of the aspects or examples of the present disclosure.

Aspects and examples of the present disclosure thus provide a reconstruction method, associated training method, and corresponding reconstruction and training nodes, that use a shared Neural Field to predict a field of attenuation coefficients, and hance density, from CBCT projections of a patient. The shared NF is shared between, and can be used to produce reconstructions for, multiple different patients. The shared NF is modulated, or conditioned, for individual patients by a patient specific NF. By sharing a conditional NF over scans taken from different patients, methods according to the present disclosure avoid retraining a reconstruction NF (that predicts the field of attenuation coefficients) from scratch for each patient, so saving considerable time and associated memory and compute resources. The patient specific NF offers an effective local modulation to condition the shared NF to each patient without requiring excessive time or computation resources. Methods according to the present disclosure offer improved performance, reconstructing with good tissue contrast and not overfitting noise. In addition, examples of the present disclosure represent an efficient improvement over previous approaches, in terms of GPU memory, scalability, and reconstruction quality.

### BRIEF DESCRIPTION OF THE DRAWINGS

For a better understanding of the present disclosure, and to show more clearly how it may be carried into effect, reference will now be made, by way of example, to the following drawings in which:

FIG. 1 is a flow chart illustrating process steps in a computer implemented method for reconstructing a volumetric medical image of a patient from CBCT projections of the patient;

FIGS. 2a to 2c show flow charts illustrating a further example of a method for reconstructing a volumetric medical image of a patient from CBCT projections of the patient;

FIGS. 3a to 3c show flow charts illustrating process steps in a computer implemented method for training a shared Neural Field for use in reconstructing a volumetric medical image of a patient from CBCT projections of the patient;

FIGS. 4a and 4b are block diagrams illustrating functional units in an example reconstruction node and an example training node respectively;

FIG. 5 illustrates a framework implementing an example method for reconstructing a volumetric medical image of a patient from CBCT projections of the patient;

FIG. 6 illustrates multi-resolution hash encoding;

FIG. 7 shows Table 1, illustrating results of comparative experiments;

FIGS. 8, 9 and 10 illustrate ground truth and reconstructions for the comparative experiments;

FIG. 11 illustrates, for the comparative examples, the percentage of the best PSNR that a model can reach over the number of steps required to achieve it using noisy projections.

### DETAILED DESCRIPTION

Certain Computed Tomography (CT) methods require large numbers of noise-free projections for accurate density reconstructions, limiting their applicability to the more complex class of Cone Beam Geometry CT (CBCT) reconstruction. As discussed above, deep learning methods can help overcome these limitations, with methods based on neural fields (NF) showing strong performance. As discussed in more detail below, Neural Fields seek to approximate the reconstructed density through a continuous-in-space coordinate-based neural network. The methods proposed in the present disclosure can help improve on Neural Field approaches. Unlike certain other approaches, which require training an NF from scratch for each new set of projections (i.e., each new patient), methods of the present disclosure can leverage anatomical consistencies over scans of different patients by training a single conditional NF on a dataset of projections. This conditional NF is shared, in the sense that it is not specific to any one patient, and may be used for reconstruction on scans of multiple different patients. The present disclosure introduces a new conditioning method in which local modulations are modelled per patient as a field over the input domain through a patient specific Neural Modulation Field (NMF). The resulting process has been implemented as a framework referred to as Conditional Cone Beam Neural Tomography (CondCBNT), and, as discussed below in detail, in experiments this implementation of the methods disclosed herein shows improved performance for both high and low numbers of available projections, and on both noise-free and noisy data.

To provide additional context to the present disclosure, there now follows a brief discussion of NF approaches to volumetric image reconstruction. NFs are a class of neural architectures that parameterize a field f: [symbol] ^{d}→ [symbol] ⁿ, i.e., a quantity defined over spatial and/or temporal coordinates, using a neural network f_{θ} (see Xie et al. (2022) for a survey on NFs). In CT reconstruction, these architectures have been used to approximate patient density directly over the volume space [symbol] ³. Neural Attenuation Fields (NAF) can provide an approach to supervise NFs using only the measured attenuated photon counts at the detector. Despite showing promising results, this method requires training an NF from scratch for each volume, that is for each patient, prohibiting transfer of learned features across volumes through weight sharing, and consequently requiring significant time, memory and computation resources.

As an alternative to the above approach, one can encode a set of projections into a latent space shared over all training volumes, and decoding this into a density modelled as a NF. However, encoding of all available projections is only feasible when a small number of projections is used, as it would otherwise result in prohibitive compute and memory requirements.

Example methods according to the present disclosure can help avoid the use of an explicit decoder. Methods disclosed herein build on an approach to learn latent codes for a dataset of 3D shapes using auto-decoding, in which randomly initialized latent codes are optimized during training. This approach can be expanded on by using these learned latent codes as modulations for a shared NF. It can be shown that the use of a single global code per signal limits reconstruction quality, and instead use a spatially structured grid of codes. Their approach greatly increases reconstruction quality, but requires interpolating a grid of modulations, increasing computational requirements for signals over higher-dimensional domains. The present disclosure introduces the Neural Modulation Field (NMF), which models a continuous field of modulations over the signal domain. This use of a patient specific NMF to modulate a conditional NF that models patient density over volume can be implemented in the architecture presented herein as the CondCBNT framework. This framework incorporates the local conditioning function modelled by the NMF to speed up reconstruction, while still processing all available projections, so relieving restrictions on projection counts used in the reconstruction process. The CondCBNT framework has been shown in experiments to provide considerable improvements in scenarios with both sufficient or limited projections, as well as in the presence of both noisy and noise-free data. These experiments are presented in greater detail later in the present disclosure.

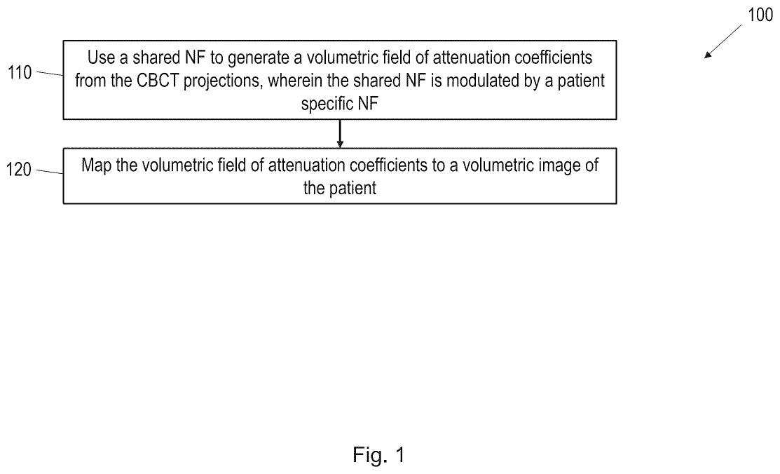

FIG. 1 is a flow chart illustrating process steps in a computer implemented method 100 for reconstructing a volumetric medical image of a patient from CBCT projections of the patient. The method may be performed by a reconstruction node, which may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The reconstruction node may encompass multiple logical entities, as discussed in greater detail below.

Referring to FIG. 1, the method 100 comprises, in a first step 110 using a shared Neural Field (NF) to generate a volumetric field of attenuation coefficients from the CBCT projections, wherein the shared NF is modulated by a patient specific NF. The method further comprises, in step 120, mapping the volumetric field of attenuation coefficients to a volumetric image of the patient.

The method 100 thus reconstructs a patient volume from CBCT projections using a shared Neural Field (examples of which may be referred to in the present disclosure as a Conditional Neural Field (CNF)) that is modulated by a patient specific Neural Field (examples of which may be referred to in the present disclosure as a Neural Modulation Field (NMF)). It will be appreciated that unlike certain other approaches using NFs, according to examples of the present disclosure, the shared NF can be shared between patients, i.e., may be used for reconstruction of volumes of multiple different patients, and is modulated for a specific patient by a second, patient specific NF, which is unique to a given patient. This avoids the need to retrain the reconstruction NF (that predicts the field of attenuation coefficients) from scratch for each patient. While different approaches to modulation of NFs are available, examples of the present disclosure propose the use of a patient specific NF, which models a continuous field of modulations over the signal domain, and thus offers an effective local modulation to condition the shared NF to each patient without requiring excessive time or computation resources. It will be appreciated that the method 100 relates to an inference phase, in which the shared NF, conditioned by the patient specific NF, is used to reconstruct a patient volume. This may be complemented by a method relating to a training phase, which is discussed in greater detail below.

As discussed above, a NF is a neural architecture that parameterizes a field, i.e., a quantity defined over spatial and/or temporal coordinates, using a neural network. A neural network is an example of a Machine Learning (ML) model. For the purposes of the present disclosure, the term “ML model” encompasses within its scope the following concepts:
machine Learning algorithms, comprising processes or instructions through which data may be used in a training process to generate a model artefact for performing a given task, or for representing a real-world process or system; and
the model artefact that is created by such a training process, and which comprises the computational architecture that performs the task.

Modulation refers to a process in which the computation carried out by a Machine Learning model is conditioned, or influenced, by information extracted from an auxiliary source. The conditioning may take the form of one or more transformations applied to a model, for example to the weights or activations of a neural network.

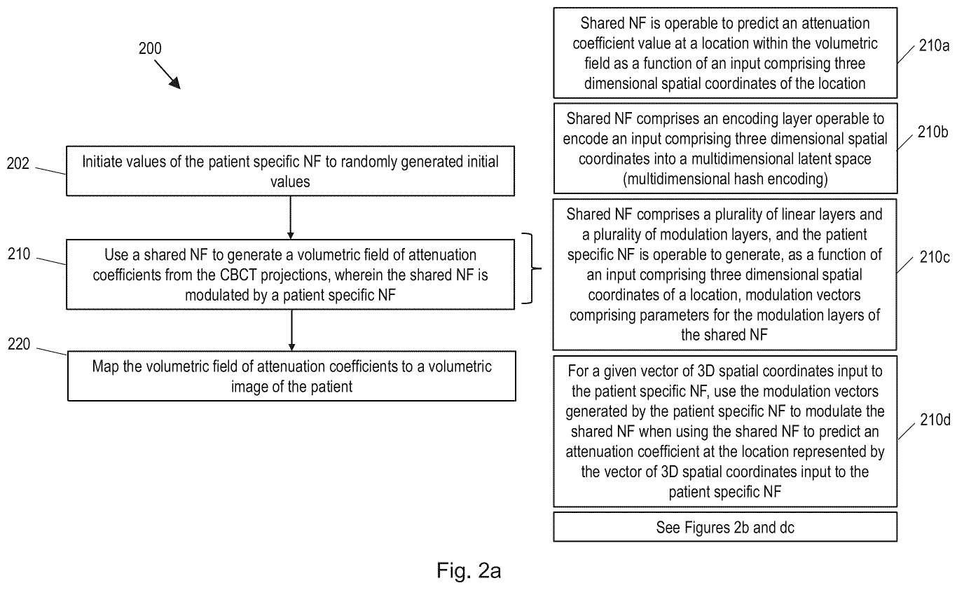

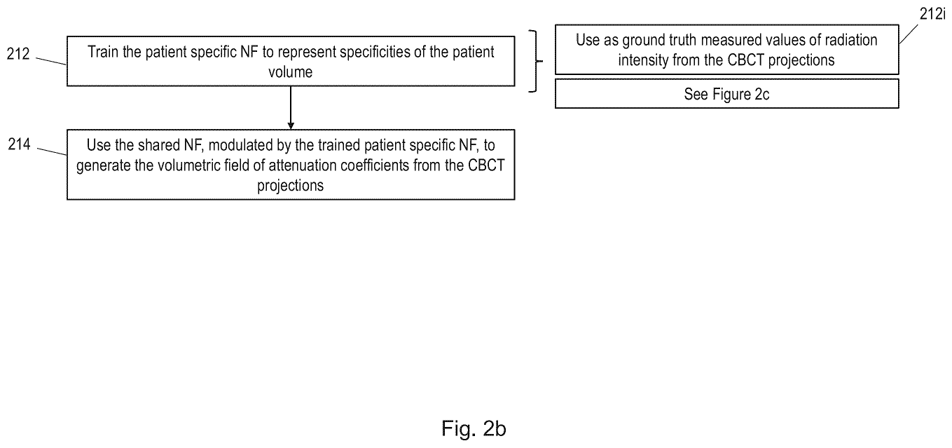

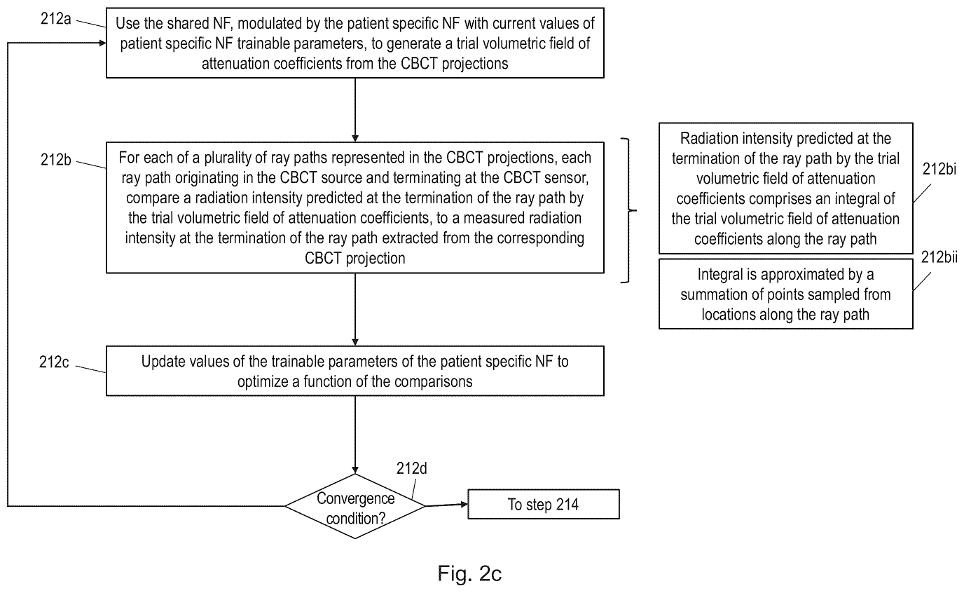

FIGS. 2a to 2c show flow charts illustrating a further example of a method 200 for reconstructing a volumetric medical image of a patient from CBCT projections of the patient. As for the method 100 discussed above, the method 200 may be performed by a reconstruction node, which may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The reconstruction node may encompass multiple logical entities, as discussed in greater detail below. The method 200 illustrates an example of how the steps of the method 100 may be implemented and supplemented to provide the above discussed and additional functionality.

Referring initially to FIG. 2a, the reconstruction node may first initiate values of the patient specific NF to randomly generated initial values in step 202. The reconstruction node then uses a shared Neural Field (NF) to generate a volumetric field of attenuation coefficients from the CBCT projections in step 210, wherein the shared NF is modulated by a patient specific NF. As illustrated at 210a, the shared NF may be operable to predict an attenuation coefficient value at a location within the volumetric field as a function of an input comprising three dimensional spatial coordinates of the location. As illustrated at 210b, the shared NF may comprise an encoding layer operable to encode an input comprising three dimensional spatial coordinates into a multidimensional latent space. In some examples, the dimensions of the latent space may be >>3. In some examples, the encoding layer may implement multiresolution hash encoding.

As illustrated at 210c, the shared NF may comprise a plurality of linear layers and a plurality of modulation layers, and the patient specific NF may be operable to generate, as a function of an input comprising three dimensional spatial coordinates of a location, modulation vectors comprising parameters for the modulation layers of the shared NF.

In some examples, the first linear layer may process the output of the encoding layer, with each linear layer being modulated by one or more modulation layers. The modulation layers may be Feature wise Linear Modulations (FiLM) layers, and the modulation vectors may be a shifting vector and a scaling vector.

As illustrated at 210d, using the shared NF to generate a volumetric field of attenuation coefficients from the CBCT projections may comprise, for a given vector of 3D spatial coordinates input to the patient specific NF, using the modulation vectors generated by the patient specific NF to modulate the shared NF when using the shared NF to predict an attenuation coefficient at the location represented by the vector of 3D spatial coordinates input to the patient specific NF.

According to examples of the present disclosure, the shared NF and patient specific NF may thus share the same input, with the patient specific NF modelling a field of modulations over the spatial coordinates of the patient volume, and the shared NF modelling a field of attenuation coefficients over the spatial coordinates of the patient volume. By inputting the same vector of spatial coordinates to each NF, the correct modulations for a given location may be used to condition the output of the shared NF to the patient under consideration.

The reconstruction node then maps the volumetric field of attenuation coefficients to a volumetric image of the patient in step 220.

FIGS. 2b and 2c illustrate additional process steps that may be carried out in order to implement the step 210 of using a shared NF to generate a volumetric field of attenuation coefficients from the CBCT projections, wherein the shared NF is modulated by a patient specific NF.

Referring now to FIG. 2b, using the shared NF to generate a volumetric field of attenuation coefficients from the CBCT projections may comprise training the patient specific NF to represent specificities of the patient volume in step 212, before using the shared NF, modulated by the trained patient specific NF, to generate the volumetric field of attenuation coefficients from the CBCT projections in step 214. Thus, the inference phase embodied in the method 200 may encompass inference phase training of the patient specific NF, before the shared NF is used to reconstruct the patient volume.

As illustrated at 212i, training the patient specific NF to represent specificities of the patient volume may comprise using as ground truth measured values of radiation intensity from the CBCT projections. Steps that may be carried out in order to implement this inference phase training of the patient specific NF are illustrated in FIG. 2c.

Referring now to FIG. 2c, training the patient specific NF to represent specificities of the patient volume may comprise repeating steps 212a to 212c illustrated in FIG. 2c until a convergence condition is satisfied. Step 212a comprises using the shared NF, modulated by the patient specific NF with current values of patient specific NF trainable parameters, to generate a trial volumetric field of attenuation coefficients from the CBCT projections. The first time step 212a is performed, the current values of the trainable parameters of the patient specific NF may be the randomly initialized values of step 202. In subsequent repetitions of step 212a, the current values will be those as updated in the most recent execution of step 212c discussed below.

Step 212b comprises, for each of a plurality of ray paths represented in the CBCT projections, each ray path originating in the CBCT source and terminating at the CBCT sensor, comparing a radiation intensity predicted at the termination of the ray path by the trial volumetric field of attenuation coefficients, to a measured radiation intensity at the termination of the ray path extracted from the corresponding CBCT projection.

As illustrated at 212bi, the radiation intensity predicted at the termination of the ray path by the trial volumetric field of attenuation coefficients may comprise an integral of the trial volumetric field of attenuation coefficients along the ray path. As illustrated at 212bii, the integral may be approximated by a summation of points sampled from locations along the ray path.

Step 212c comprises updating values of the trainable parameters of the patient specific NF to optimize a function of the comparisons. Different options may be envisaged for the function of the comparisons. For example, the Mean Square Error (MSE) may be calculated between the predicted and measured intensity at the termination of the ray path. Other examples of loss or cost function may also be considered. In some examples, optimizing the function of the comparisons may comprise minimizing the function. Optimization methods, including for example gradient descent, may be used for carrying out step 212c.

In step 212d, the reconstruction node checks whether the convergence criterion has been satisfied for the training of the patient specific NF. This convergence criterion may for example comprise a threshold value of the function of the comparisons (e.g., a threshold MSE), a threshold number of training iterations, a maximum or minimum training time, etc. A combined criterion may also be envisaged, in which some combination of threshold function value, training time and number or iterations is used, for example imposing a training stop after a certain maximum time, in the event that a threshold value for the function has not already been reached.

If the convergence criterion has been satisfied, then the reconstruction node advances to step 214, and uses the shared NF, modulated by the trained patient specific NF, to generate the volumetric field of attenuation coefficients from the CBCT projections. If the convergence criterion has not yet been satisfied, then the reconstruction node returns to step 212a.

The methods 100, 200 may be complemented by a method for training the shared NF that is used in the methods 100 and 200.

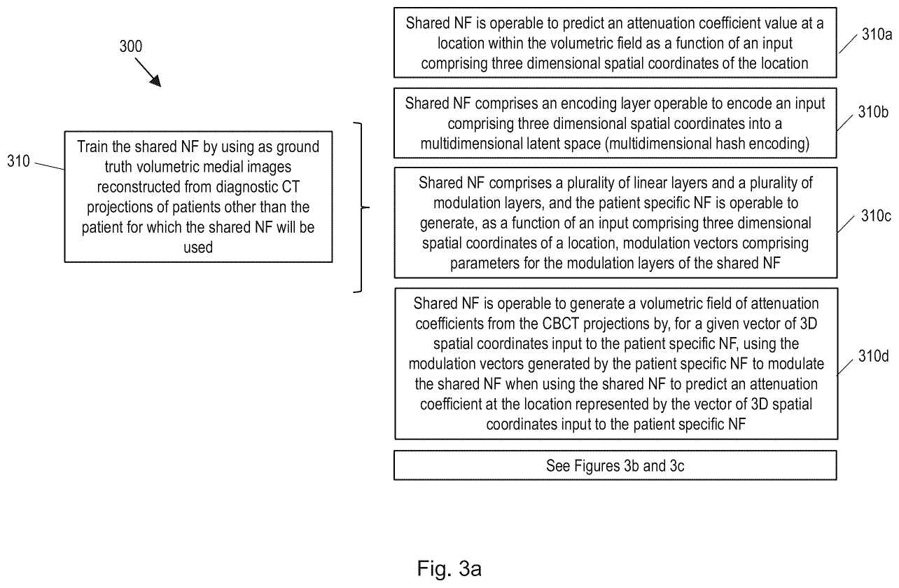

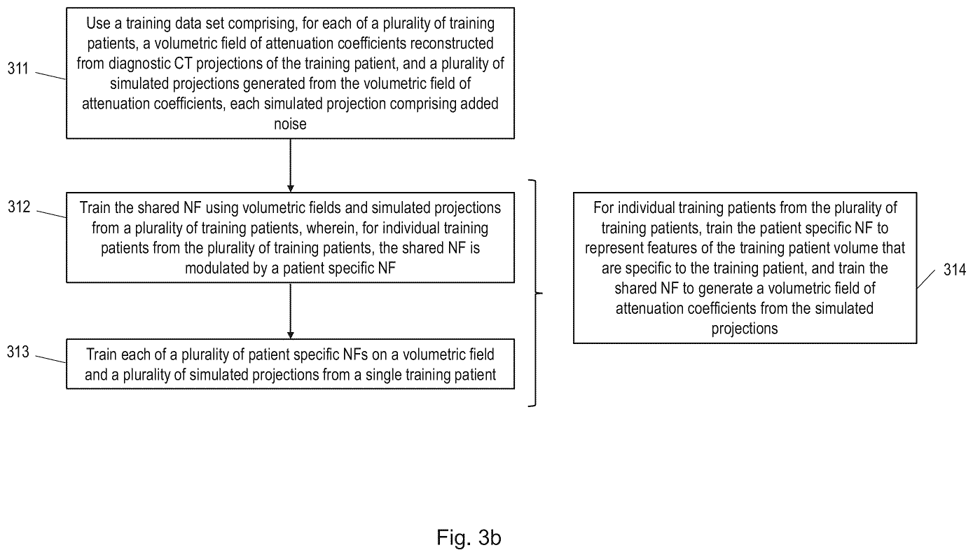

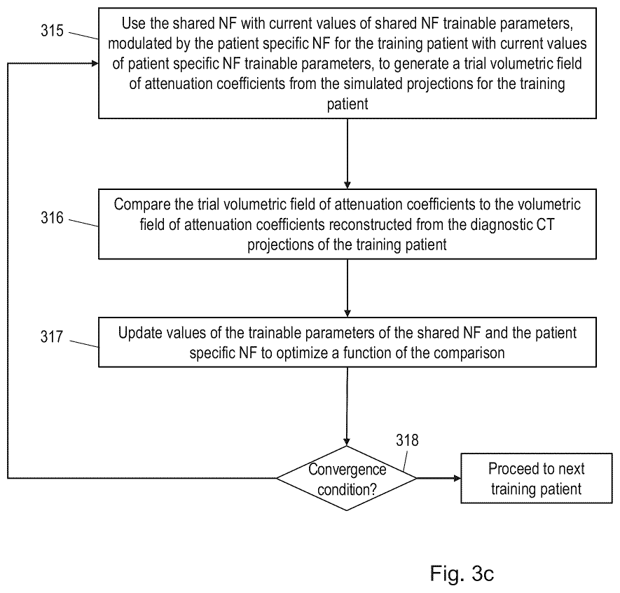

FIGS. 3a to 3c show flow charts illustrating process steps in a computer implemented method 300 for training a shared Neural Field for use in reconstructing a volumetric medical image of a patient from CBCT projections of the patient, wherein the shared NF is operable to generate a volumetric field of attenuation coefficients from the CBCT projections, and wherein the shared NF is modulated by a patient specific NF. The method may be performed by a training node, which may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The training node may encompass multiple logical entities, as discussed in greater detail below.

Referring initially to FIG. 3a the method 300 comprises, in step 310, training the shared NF by using as ground truth volumetric medial images reconstructed from diagnostic CT projections of patients other than the patient for which the shared NF will be used. It will be appreciated that for the purposes of the present specification, diagnostic CT projections, which may also be referred to as planning CT projections, are projections obtained using what may be understood as a conventional CT scanner, and not a CBCT scanner. Briefly, a conventional CT scanner uses a rotating, high-output anode X-ray tube and records data with a fan-shaped beam onto image detectors placed in an are around the patient. A CBCT scanner uses a cone-shaped beam radiating from an X-ray source that covers a large volume with a single rotation, and records data onto a panel detector that also rotates around the patient. Owing to the differences between CT and CBCT scanning, volumetric medial images reconstructed from CT projections of patients are generally of higher image quality than those reconstructed from CBCT projections. By training on these higher quality reconstructions, the shared NF can learn to recognize what the desirable level of quality is for the CBCT reconstructions.

As illustrated at 310a, the shared NF may be operable to predict an attenuation coefficient value at a location within the volumetric field as a function of an input comprising three dimensional spatial coordinates of the location.

As illustrated at 310b, the shared NF may comprise an encoding layer operable to encode an input comprising three dimensional spatial coordinates into a multidimensional latent space. In some examples, the encoding layer may implement multiresolution hash encoding.

As illustrated at 310c, the shared NF may comprise a plurality of linear layers and a plurality of modulation layers, and the patient specific NF may be operable to generate, as a function of an input comprising three dimensional spatial coordinates of a location, modulation vectors comprising parameters for the modulation layers of the shared NF.

As illustrated at 310d, the shared NF may be operable to generate a volumetric field of attenuation coefficients from the CBCT projections by, for a given vector of 3D spatial coordinates input to the patient specific NF, using the modulation vectors generated by the patient specific NF to modulate the shared NF when using the shared NF to predict an attenuation coefficient at the location represented by the vector of 3D spatial coordinates input to the patient specific NF.

FIGS. 3b and 3c illustrate additional process steps that may be carried out in order to implement the step 310 of training the shared NF by using as ground truth volumetric medial images reconstructed from diagnostic CT projections of patients other than the patient for which the shared NF will be used.

Referring initially to FIG. 3b, and as illustrated at step 311, training the shared NF may further comprise using a training data set comprising, for each of a plurality of training patients, a volumetric field of attenuation coefficients reconstructed from diagnostic CT projections of the training patient, and a plurality of simulated projections generated from the volumetric field of attenuation coefficients, each simulated projection comprising added noise.

Training the shared NF may further comprise, in step 312, training the shared NF using volumetric fields and simulated projections from a plurality of training patients, wherein, for individual training patients from the plurality of training patients, the shared NF is modulated by a patient specific NF. Training the shared NF may also comprise, in step 313, training each of a plurality of patient specific NFs on a volumetric field and a plurality of simulated projections from a single training patient. In this manner, the training may be adapted to ensure simultaneous training of both the shared NF and multiple patient specific NFs.

As illustrated at step 314, training the shared NF may further comprise, for individual patients of the plurality of training patients, training the patient specific NF to represent features of the training patient volume that are specific to the training patient, and training the shared NF to generate a volumetric field of attenuation coefficients from the simulated projections.

By training a different patient specific NF for each patient, not only do the patient specific NFs learn to represent the particularities of individual patients, but the shared NF learns to recognize what is consistent across patients, and so to reconstruct accurately the features common to all patients. The patient specific NFs that are developed in training may in some examples not actually be used in the inference phase, but their purpose is to shape the training of the shared NF.

Referring now to FIG. 3c, training the shared NF may further comprise, for individual training patients from the plurality of training patients, repeating steps 315 to 317 until a convergence condition is satisfied.

Step 315 comprises using the shared NF with current values of shared NF trainable parameters, modulated by the patient specific NF for the training patient with current values of patient specific NF trainable parameters, to generate a trial volumetric field of attenuation coefficients from the simulated projections for the training patient.

Step 316 comprises comparing the trial volumetric field of attenuation coefficients to the volumetric field of attenuation coefficients reconstructed from the diagnostic CT projections of the training patient.

Step 317 comprises updating values of the trainable parameters of the shared NF and the patient specific NF to optimize a function of the comparison.

It will be appreciated that the training procedure outlined in steps 315 to 317 differs from the inference phase training of the patient specific NF which may be carried out as part of the method 200, and is described above and illustrated in FIG. 2c. In the method 300, the ground truth is the diagnostic CT reconstructions of training patients, and parameters of both the shared NF and patient specific NFs are updated. In the method 200, the patient specific NF is first trained on that specific patient using line integrals over the predicted field, supervised by the measured intensity at the detector.

As discussed above for the patient specific training of the patient specific NF, different options may be envisaged for the function of the comparison in step 317. For example, the Mean Square Error (MSE) may be calculated between the trial volumetric field of attenuation coefficients to the volumetric field of attenuation coefficients reconstructed from the diagnostic CT projections of the training patient. Other examples of loss or cost function may also be considered. In some examples, optimizing the function of the comparison may comprise minimizing the function. Optimization methods, including for example gradient descent, may be used for carrying out step 317.

In step 318, the training node checks whether the convergence criterion has been satisfied for the training of the shared NF. This convergence criterion may for example comprise a threshold value of the function of the comparison (e.g., a threshold MSE), a threshold number of training iterations, a maximum or minimum training time, etc. A combined criterion may also be envisaged, in which some combination of threshold function value, training time and number or iterations is assembled.

If the convergence criterion has been satisfied, then the training node advances proceeds to repeat steps 315 to 317 for a next training patient. If the convergence criterion has not yet been satisfied, then the training node returns to step 315.

In some examples of the method 300, for individual training patients from the plurality of training patients, values of trainable parameters for the shared NF may be initiated to the values following training on a previous training patient.

In some examples of the method 300, for individual training patients from the plurality of training patients, values of trainable parameters for the patient specific NF may be initiated to randomly generated initial values.

Example methods according to the present disclosure achieve CBCT volume reconstruction that offers both speed and reconstruction quality. The methods described above offer speed advantages associated with not having to retrain the NF used for prediction of attenuation coefficients (hence density) from scratch for each patient. In addition, the methods proposed herein offer improved image quality, through the use of Neural Fields for modelling patient structures, and through the use of the patient specific NF, which offers effective local conditioning to each unique patient. This combination of speed and quality can support both the planning and delivery of radiotherapy treatment, for example in the form of online Adaptive Radiotherapy (ART).

The speed and quality afforded by methods of the present disclosure can support the provision of online ART, in which in which CBCT is used to capture patient imaging at the start of each visit of the treatment fraction. This up-to-date imaging data, if available with sufficient quality, can enable clinicians to track changes in patient anatomy, including for example tumour shrinkage over the course of the radiotherapy treatment, allowing for online target localisation and plan adaptation without the constraints of diagnostic CT imaging. The improved image quality offered by methods according to the present disclosure may result in many additional medical treatment benefits (including improved accuracy of radiotherapy treatment, reduced exposure to unintended radiation, reduced treatment duration, etc.). The methods presented herein may be applicable to a variety of medical treatment and diagnostic settings or radiotherapy treatment equipment and devices.

In one particular use case for methods of the present disclosure, a dose from a previous treatment session can be deformed or modified in light of the current patient anatomy as represented by the reconstructed volumetric image of the patient. The output of the methods disclosed herein may thus be used in the creation or adaptation of a radiotherapy treatment plan.

As discussed above, the methods 100 and 200 may be performed by a reconstruction node, and the present disclosure provides a reconstruction node that is adapted to perform any or all of the steps of the above discussed methods. The reconstruction node may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The reconstruction node may encompass multiple logical entities, as discussed in greater detail below.

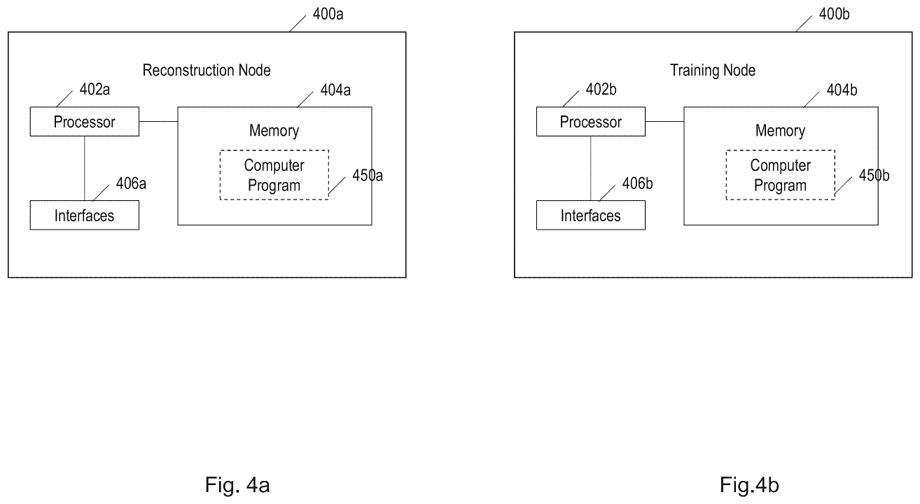

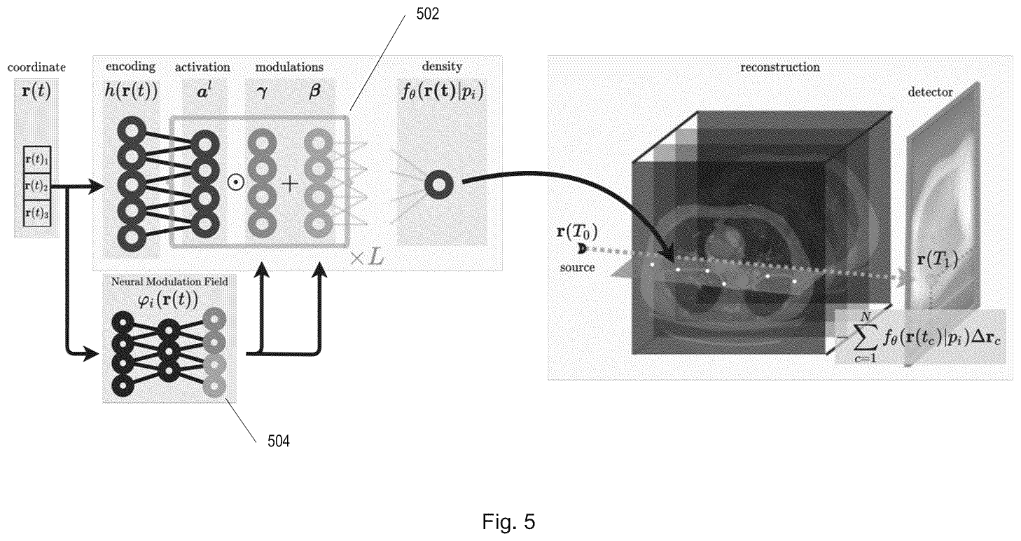

FIG. 4a is a block diagram illustrating an example reconstruction node 400a which may implement the method 100 and/or 200, as illustrated in FIGS. 1 to 2c, according to examples of the present disclosure, for example on receipt of suitable instructions from a computer program 450a. Referring to FIG. 4a the reconstruction node 400a comprises a processor or processing circuitry 402a, and may comprise a memory 404a and interfaces 406a. The processing circuitry 402a is operable to perform some or all of the steps of the method 100 and/or 200 as discussed above with reference to FIGS. 1 to 2c. The memory 404a may contain instructions executable by the processing circuitry 402a such that the reconstruction node 400a is operable to perform some or all of the steps of the method 100 and/or 200, as illustrated in FIGS. 1 to 2c. The instructions may also include instructions for executing one or more telecommunications and/or data communications protocols. The instructions may be stored in the form of the computer program 450a. In some examples, the processor or processing circuitry 402a may include one or more microprocessors or microcontrollers, as well as other digital hardware, which may include digital signal processors (DSPs), special-purpose digital logic, etc. The processor or processing circuitry 402a may be implemented by any type of integrated circuit, such as an Application Specific Integrated Circuit (ASIC), Field Programmable Gate Array (FPGA) etc. The memory 404a may include one or several types of memory suitable for the processor, such as read-only memory (ROM), random-access memory, cache memory, flash memory devices, optical storage devices, solid state disk, hard disk drive, etc.

In some examples as discussed above, the reconstruction node may be incorporated into treatment apparatus, and examples of the present disclosure also provide a treatment apparatus comprising either or both of a reconstruction node as discussed above and/or a planning node operable to implement a method for adapting a radiotherapy treatment plan.

As discussed above, the method 300 may be performed by a training node, and the present disclosure provides a training node that is adapted to perform any or all of the steps of the above discussed method. The training node may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The training node may encompass multiple logical entities, as discussed in greater detail below.

FIG. 4b is a block diagram illustrating an example training node 400b which may implement the method 300, as illustrated in FIGS. 3a to 3c, according to examples of the present disclosure, for example on receipt of suitable instructions from a computer program 450b. Referring to FIG. 4b the training node 400b comprises a processor or processing circuitry 402b, and may comprise a memory 404b and interfaces 406b. The processing circuitry 402b is operable to perform some or all of the steps of the method 300 as discussed above with reference to FIGS. 3a to 3c. The memory 404b may contain instructions executable by the processing circuitry 402b such that the training node 400b is operable to perform some or all of the steps of the method 300, as illustrated in FIGS. 3a o 3c. The instructions may also include instructions for executing one or more telecommunications and/or data communications protocols. The instructions may be stored in the form of the computer program 450b. In some examples, the processor or processing circuitry 402b may include one or more microprocessors or microcontrollers, as well as other digital hardware, which may include digital signal processors (DSPs), special-purpose digital logic, etc. The processor or processing circuitry 402b may be implemented by any type of integrated circuit, such as an Application Specific Integrated Circuit (ASIC), Field Programmable Gate Array (FPGA) etc. The memory 404b may include one or several types of memory suitable for the processor, such as read-only memory (ROM), random-access memory, cache memory, flash memory devices, optical storage devices, solid state disk, hard disk drive, etc.

In some examples as discussed above, the training node may be incorporated into a treatment apparatus, and examples of the present disclosure also provide a treatment apparatus comprising either or both of a reconstruction node and/or a training node as discussed above.

FIGS. 1 to 3c discussed above provide an overview of methods which may be performed according to different examples of the present disclosure. These methods may be performed by a reconstruction node and a training node respectively, as illustrated in FIGS. 4a and 4b.

There now follows a detailed discussion of how different process steps illustrated in FIGS. 1 to 3c and discussed above may be implemented, as well as presentation of experimental results for the example implementation. The functionality and implementation detail described below is discussed with reference to the modules of FIGS. 4a and 4b performing examples of the methods 100, 200 and/or 300, substantially as described above. In the following discussion, for convenience, reference is made to the shared NF modelling patient density over volume, as opposed to modelling attenuation coefficients. It will be appreciated that this reference to modelling density encompasses the modelling of attenuation coefficients, given the strong relation between them under the assumptions that hold for the CBCT setting.

FIG. 5 illustrates a framework implementing an example of the methods disclosed herein. The framework illustrated in FIG. 5 is referred to in the following disclosure as Conditional Cone Beam Neural Tomography (CondCBNT). As discussed above, CondCBNT is for reconstructing CBCT volumes using NFs, and comprises a conditional NF 502 and a patient specific NF 504. FIG. 5 illustrates an inference phase, in which examples of the methods 100, 200 are carried out. During this phase, coordinates r(t) from a volume surrounding the patient are encoded by the shared NF into a multiresolution hash-encoding h(r(t)), and passed through L linear layers. To leverage consistencies over anatomies of different patients, the density for a specific patient pᵢ is modelled using the shared neural field fᵢ, whose activations a^{l} are modulated by the patient-specific NF, or Neural Modulation Field (NMF) φᵢ. This conditioning function learns a field of γ, β FiLM modulations (Dumoulin et al., 2018) over the input space [symbol] ³ for a pᵢ. The patient specific NF learns the modulation field through calculating an integral of the predicted density along a ray path (as in steps 212bi and 212bii of method 200), and supervising the calculated integral by the measured value of radiation intensity at the termination of the path (steps 212b, 212c of method 200). An integral is taken over values sampled from the neural field f₀ at coordinates r(t) along a ray cast from source to sensor. This integral:

$$- {\sum }_{c = 1}^{N} {f}_{θ} ( r ( {t}_{c} ) ) Δ {r}_{c}$$

is supervised at the sensor using the corresponding observed projection value.

Beer-Lambert's law relates the attenuation of electromagnetic radiation such as visible light or X-rays to the properties of the material it is traveling through (Swinehart, 1962). Let r: [T₀, T₁]→ [symbol] ³ be the straight path taken by radiation through the medium. The radiation intensity I(r(T₁)) at position r(T₁) is the line integral:

$$I ( r ( {T}_{1} ) ) = {I}_{0} exp [ - {\int }_{{T}_{0}}^{{T}_{1}} μ ( r ( t ) ) \vert {r}^{′} ( t ) \vert dt ] \qquad Equation 1$$

where μ: [symbol] ³→ [symbol] ⁺ is the attenuation coefficient of the medium and I₀=I(r(T₀)) is the initial intensity. The integral in Equation 1 can be approximated by the sum:

$$I ( r ( {T}_{1} ) ) \approx {I}_{0} exp [ - {\sum }_{c = 1}^{N} μ ( r ( {t}_{c} ) ) \vert {r}^{′} ( {t}_{c} ) \vert Δ t ] \qquad Equation 2$$

where t_{c} ∈ [T₀,T₁] and |r′(t_{c})|Δt=Δr_{c}=|r(t_{c+1})−r(t_{c})|. Given a set of 2D CBCT projections vₐ ∈ [symbol] ^{H×W} with H, W the height and width of the sensor and a the angle under which the projection was taken, during training of the patient specific modulating NF, the goal is to estimate density values along rays cast from source to sensor. Each ray is the straight path r which connects the source to pixels in the detector. For simplicity, the patient volume is bound with a box and zero attenuation is assumed outside the box. Therefore, for every path, the sum in Equation 2 is computed with only those r(t_{c}) that are contained in the bounding box. By taking the logarithm, the computationally tedious exponential can be avoided, and the following expression can be used:

$$log I ( r ( {T}_{1} ) ) \approx - {\sum }_{c = 1}^{N} μ ( r ( {t}_{c} ) ) Δ {r}_{c} + log {I}_{0}$$

It is also possible to discard the constant that depends on the initial intensity, which it may be assumed is the same for all projections. A neural field f₀: [symbol] ³→ [symbol] ⁺ is used to approximate the density μ such that the intensity I(r(T₁) coincides with the intensity recorded by the detector at the position r(T₁). The neural field is parameterised by the shared NF.

$$log I ( r ( {T}_{1} ) ) \approx - {\sum }_{c = 1}^{N} {f}_{θ} ( r ( {t}_{c} ) ) Δ {r}_{c} \qquad Equation 3$$

As discussed above, CondCBNT first embeds coordinates into a multidimensional latent space (step 210b of method 200). It can be shown that ReLU MLPs suffer from spectral bias, limiting their capacity to model high frequency functions on low-dimensional domains. As a solution, it is possible to embed coordinates r(t_{c}) ∈ [symbol] ³ into a higher-dimensional space [symbol] ^{e} with e>>3 before passing them through the MLP. The CondCBNT implementation of the methods disclosed herein can use the multiresolution hash-encoding, denoted h(r(tᵢ), as it empirically shows fastest convergence in the experiments set out below. There now follows a description of this embedding.

Multi-resolution hash encoding can be used to help coordinate embedding for neural fields. This method is now briefly described in more detail. Multi-resolution hash encoding is a parametric embedding, meaning the embedding function itself contains additional trainable parameters. In multi-resolution hash encoding this is done through assigning freely trainable weights to grid points from a set of multi-resolution grids defined over the input space. These parameters are then looked up and interpolated for a specific input coordinate x. Formally, the embedding consists of a number of levels L, which correspond to the multiple grid resolutions, a feature dimensionality d denoting the dimensionality of each trainable vector attached at a grid point, a base resolution denoting the number of grid points for the lowest resolution grid, a per-level resolution increase factor r, and a maximum hash-table size.

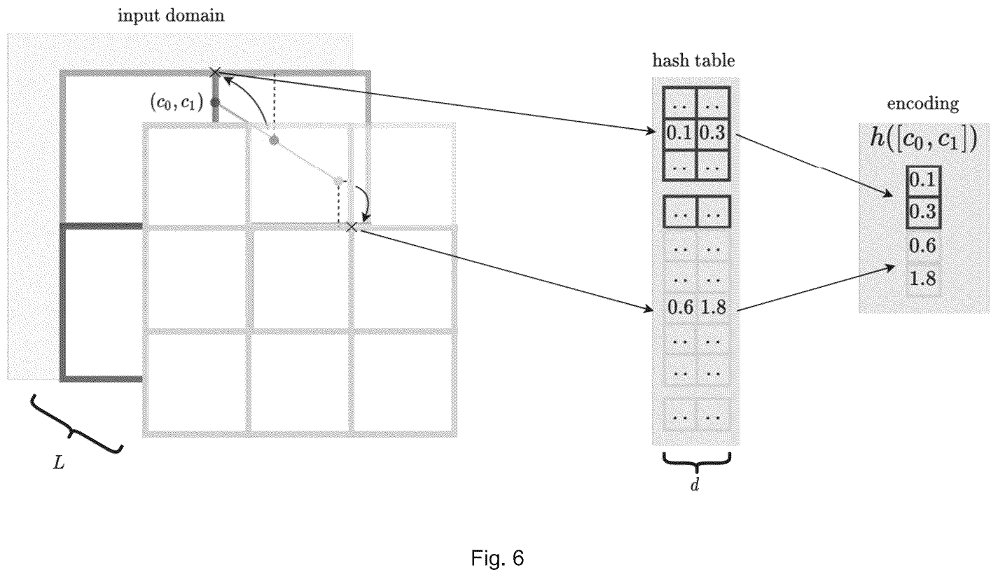

FIG. 6 illustrates multi-resolution hash encoding. L grids of multiple resolutions (green, red) are defined over the input domain. Each grid-point corresponds to an entry in a hash table. Each entry in this hash table consists of a d dim freely trainable weight vector. To encode a coordinate (c₀, cₗ), it is mapped to its closest grid-points on every grid (in practice a linear interpolation of the n nearest grid points is taken), and the encoding for this coordinate is given by concatenating the grid points' corresponding feature vectors, to end up with a (L·d)−dimensional embedding.

Methods according to examples of the present disclosure propose to condition the shared NF with patient specific Neural Modulation Fields. Conditioning in neural fields consists of modulating the weights θ or activations a of a NF f₀ with a conditioning variable z to vary the NF's output. This method may be used to encode different samples xᵢ from a single dataset X through a set of latents {zᵢ|xᵢ ∈ X}. In the setting of CBCT reconstruction, it may be assumed that the densities for patients pᵢ ∈ P share a lot of anatomical structure. A conditional NF that is tasked with reconstructing a dataset of multiple volumes would be able to leverage this consistency in anatomical information in its reconstruction (e.g., inferring from noisy or missing data), with patient-specific characteristics being refined with the conditioning variable zᵢ. It could therefore be envisaged, in principle, to use the aforementioned auto-decoding approach with a global conditioning latent zᵢ. However, global conditioning can be shown to result in reconstructions with limited detail. This limitation is significant because patient-specific fine-grained details in scans contain information crucial for medical purposes including treatment planning and adaptation in Radiotherapy and Adaptive Radiotherapy.

Examples of the present disclosure use local conditioning, in which the conditioning variable zᵢ depends on the input coordinate r(t). In previous works, local conditioning has been achieved through interpolation of a trainable discrete data structure, e.g., a grid of latent codes. In contrast to this approach, and in order to further increase expressivity of the resulting modulation and forego modelling choices such as code grid resolution and interpolation method, the present disclosure proposes to abstract the learning of modulations away from a discrete data structure and model the modulations themselves as a continuous field through a patient-specific Neural Modulation Field (NMF) denoted φᵢ. During training (as in the method 300), parameters θᵢ of the patient-specific NMFs φ_{θ}_{ᵢ}, are optimized alongside the weights of the shared NFf_{θ} (steps 312, 313 of method 300), supervised for example by reconstructed volumes for planning or diagnostic quality CTs scans, as opposed to CBCT scans (method 300, for example step 311). During inference, when the method is presented with a new patient volume for which a planning or diagnostic CT scan is not available, the parameters of the shared NF are maintained (step 212 of method 200), and only the parameters of the patient specific NMF are updated, supervised using the integral method discussed above, in which a line integral over a ray path through the predicted density field is compared to measured intensity at the termination point of the ray path (steps 212b, 212bi, 212bii, and 212c of method 200).

For activation modulation (an examples of steps 210c and/or 210d of method 200), feature-wise linear modulations (FiLM) (Dumoulin et al., 2018) are used, such that activations a^{l} at a layer l with weights W^{l} and bias b^{l} are transformed with patient-specific local scaling and shifting modulations γᵢ, βᵢ as follows:

$${a}_{i}^{l} = ReLU ( ( {W}^{l} {a}_{i}^{l - 1} + {b}^{l} ) ⊙ {γ}_{i} + {β}_{i} ) \qquad Equation 4$$

where γᵢ, βᵢ are obtained from the NMF φ_{θ}_{ᵢ}: [symbol] ³→ [symbol] ^{dim(γ)+dim(β)}. Specific architectural choices of the NMF and shared NF of the CondCBNT implementation of the methods of the present disclosure are presented below in the context of experimental validation of this implementation of the methods disclosed herein.

### Experimental Data for CondCBNT

### Dataset

The dataset used for the experiments presented herein conforms for example to step 311 of the method 300, and is derived from the LIDC-IDRI (Armato III et al., 2015). This is a collection of diagnostic lung cancer screening thoracic CT scans. A random selection of 250 cases was chosen and the CT scan resampled to 2 mm resolution. Then, each volume was projected using 256×256 pixel, 2 mm resolution detectors. Angles equally spaced between 0° and 2050 were used. 400 projections were created, first without any noise, then with Poisson noise, used to simulate measurement noise with 5×10⁵ photons. A subset of 50 equally-spaced projections was obtained from both. The 250 volumes were split into 200/25/25 for training, validation, and testing.

### Metrics

Quantitative evaluation is provided using the Peak Signal to Noise Ratio (PSNR), a classical measure of signal quality, and the Structural Similarity Index Measure (SSIM), which captures the perceptive similarity between two images by analyzing small local chunks (Wang et al., 2004). Historically, both metrics have been defined for images, but for experimental evaluation they were computed over full volumes as discussed below. Finally, GPU memory used and time required to reconstruct a volume were also tracked.

Both PSNR and SSIM were adapted for use in a 3D setting in the following manner. Given two volumes x,y ∈ [symbol] ^{H×W×D} where H, W, and D are the height, width, and depth of the volume respectively, y is the ground truth and x is the reconstruction, the PSNR is the following

$$\begin{matrix}P S N R ( x , y ) = 10 \cdot {log}_{10} \frac{{( max y )}^{2}}{M S E ( x , y )} \\ = 20 \cdot {log}_{10} max ( y ) - 10 \cdot {log}_{10} M S E ( x , y )\end{matrix} \qquad \begin{matrix}Equation 5 \\ Equation 6\end{matrix}$$

where the second step improves numerically stability and the MSE is the voxel-wise Mean Squared Error:

$$M S E ( x , y ) = \frac{1}{NML} {\sum }_{i = 0}^{N - 1} {\sum }_{j = 0}^{M - 1} {\sum }_{k = 0}^{L - 1} {( x ( i , j , k ) - y ( i , j , k ) )}^{2} \qquad Equation 7$$

The SSIM was computed over a small K×K×K cube within the volume. This was repeated for all pixels, padding when necessary with zeros. The formula is for the entire volume, although the original definition is for a single region:

$$SSIM ( x , y ) = \frac{( 2 {μ}_{x} {μ}_{y} + {c}_{1} ) ( 2 {σ}_{x y} + {c}_{2} )}{( {μ}_{x}^{2} + {μ}_{y}^{2} + {c}_{1} ) ( {σ}_{x}^{2} + {σ}_{y}^{2} + {c}_{2}}$$

where μ indicates the mean, σ the covariance, c₁=(k₁L)² and C₂=(k₂L)² with k₁=0.01, k₂=0.03, and L the difference between the maximum value and minimum value in y.

### Baselines

The following baselines were used for comparison with CondCBNT. FDK reconstruction (Feldkamp et al., 1984) was performed using Operator Discretization Library (Adler et al., 2017). As an iterative reconstruction baseline, the Landweber iteration was implemented with Total Variation regularization (Kaipio & Somersalo, 2005), with parameters such as step size, iteration count and the amount of regularization chosen via grid search on the validation set. As a deep learning reconstruction baseline, the LIRE-32(L) architecture from Moriakov et al. (2022) was used. This is a dedicated lightweight, memory-efficient variant of learned primal-dual method from Adler & Oktem (2018) for CBCT reconstruction. From the NF class of models, CondCBNT was compared with Zha et al. (2022) (NAF). No comparison was made with Lin et al. (2023) owing to their prohibitive computational costs.

### Experiments

Hyperparameter search for NAF, CondCBNT, and the Iterative method was carried out on the validation set. With noisy projections, early stopping was used to avoid overfitting the noise. With noise-free projections, a stop was implemented after about 10 minutes of training. Although more time would have improved performance further, it would not have provided any additional insights. It should be noted that individual volume optimization was not conducted to reflect the constraints of a realistic scenario. During training, the neural field was supervised directly with density values as discussed above (for example in the context of steps 315 to 318 of the method 300), as it was observed that this greatly improved stability. During inference on validation and test sets, the shared NF was kept fixed, and only the randomly initialized NMF weights for each unseen scan were optimized (as in step 212 of the method 200).

When training NAF and CondCBNT, rays were sampled at random to form a batch. Then, a number of samples were selected along the ray to form the inputs of the model. While in NAF the batch is created using rays sampled at random from a single projection, for CondCBNT rays were sampled from any projection.

Projection noise was added using the Poisson distribution, to simulate the effect of measurement noise. This is also called shot noise, and it happens in all devices which measure the amount of photons that hit them. The probability of detecting photons can be modelled using a Poisson distribution. Intuitively, a thicker and denser substance in the path of the ray will result in a lower probability of detection and more noise in the projection. To be specific, assuming a projected value of p and a fixed photon count c (set at 5×10⁵ in the experiments), the Poisson distribution's rate is defined as λ=πe^{-P}. Thus, the probability of detecting a specific number of photons, q, can be expressed as:

$$P ( q ; λ ) = \frac{{e}^{- λ} {λ}^{q}}{q !} = \frac{{( π {e}^{- p} )}^{q} {e}^{- π {e}^{- p}}}{q !} \qquad Equation 8$$

By sampling a value q from this distribution, the resulting projected value is then calculated as:

$$\tilde{p} = - log ( \frac{q}{π} ) \qquad Equation 9$$

### Architectural Details

The architectural specifications for the shared neural field and the patient-specific modulation neural fields of CondCBNT as used in the present experiments are now described, and illustrate example implementations of steps 210a, 210b, 210c and 210d of the method 200.

The shared neural field f_{θ}, consists of a multi-resolution hash encoding, as described above, with 16 levels of feature dimensionality 2, a base resolution of 16×16×16, a per-level resolution increase factor of 2, and a hash-table with maximum size of 2¹⁹ parameters per level. This results in a 32-dimensional embedding, which is passed through 2 linear layers with hidden size 128, each followed by patient-specific FiLM modulation—as described above—and ReLU activations. Each modulation neural field φ_{θ}_{ᵢ} also uses multi-resolution hash encoding to embed the input coordinate, followed by 2 linear layers of hidden dimensionality 128 with ReLU activations, which outputs into a 2-128 dimensional code z split into γ, β∈ [symbol] ¹²⁸.

### Hyperparameters

For all experiments, the code was implemented in PyTorch (Paszke et al., 2019), optimized using Adam (Kingma & Ba, 2015) with β₁=0.9, β₂=0.999, ϵ=10⁻⁸.

CondCBNT was trained for 15 hours (an example of a time-based convergence condition) on an A100 GPU using all 200 volumes from the training set. The learning rate used for the MNF was 10⁻⁴ while 10⁻³ was used for the shared NF. During training the batch size was 16, 384. During validation and testing, the MNFs were optimized individually for each patient as in the method 200, with a batch size of 1024 rays and 300 samples along the ray. Only points within the bounding box of the patient, defined by the original CT scan, were sampled.

NAF was optimized on each volume individually, with a learning rate of 5×10⁻⁴, optimized through hyperparameter search on the validation set. For the noise-free projection settings, the model used reflected the specifications from the original paper. The hash encoding used a base resolution of 16, the maximum size of the hash table was 2²¹, the number of levels was 16 and the size of the feature vector for each level was 2. Instead, validation revealed that a base resolution of 8, with 8 levels and a hash table size of 2¹⁹ resulted in better reconstruction, as it avoided overfitting to the noise more often. For both settings, an MLP with LeakyReLU activations, 4 layers, and 32 neurons per layer was used. The batch size was also 1024 rays, with 300 points sampled per ray.

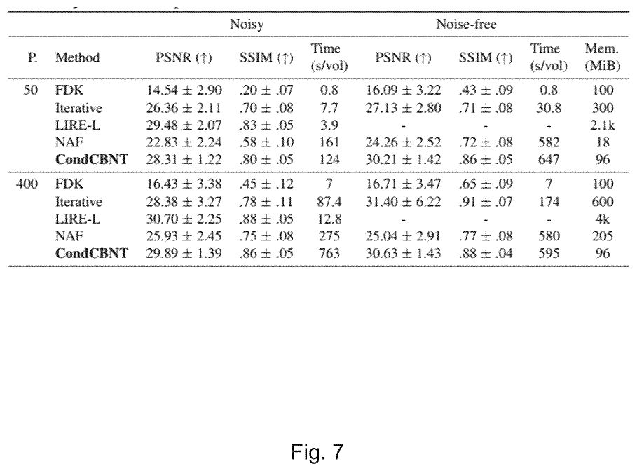

The model was first evaluated on the test set using 50 and 400 noise-free projections respectively, results shown in Table. 1 right, in FIG. 7. Table 1 illustrates mean standard deviation of metrics over test set for FDK (Feldkamp et al., 1984), Iterative (Kaipio & Somersalo, 2005), LIRE-L (Moriakov et al., 2022), NAF (Zha et al., 2022), and CondCBNT. LIRE-L slightly outperforms CondCBNT but requires more GPU memory. CondCBNT excels with less memory and comparable runtime.

CondCBNT greatly improves reconstruction quality both in terms of PSNR and SSIM, compared to classical methods and NAF. The model was then validated on 50 and 400 noisy projections, results for which are shown in Table 1 left, in FIG. 7. Again, considerable improvements are seen from CondCBNT over all baseline approaches. LIRE-L is the exception, achieving a performance slightly better than CondCBNT with significantly faster reconstruction speed at the cost of an increased memory footprint.

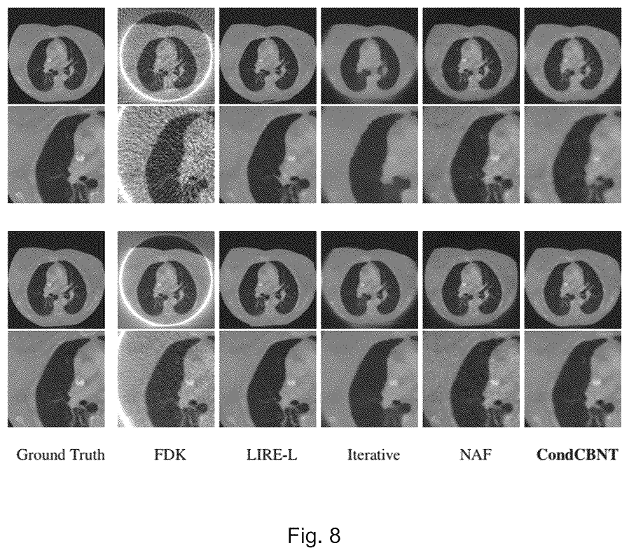

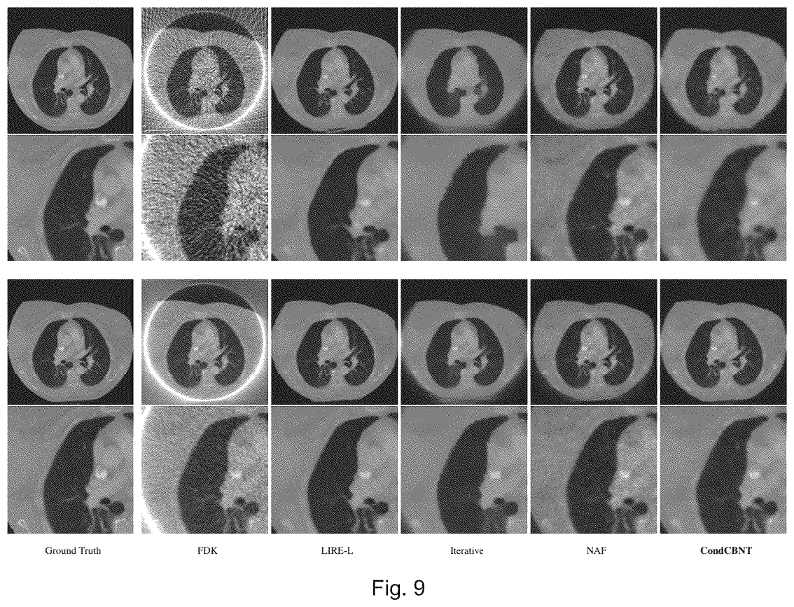

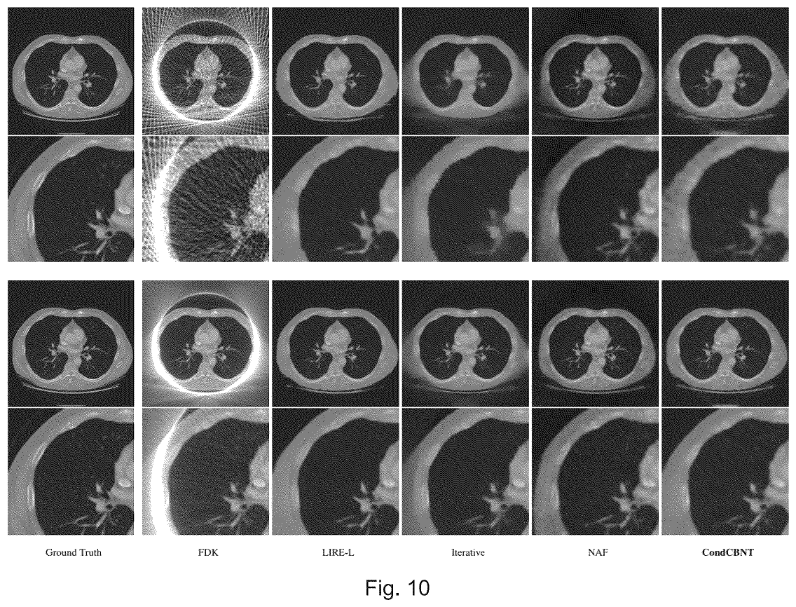

Qualitative assessment in the noisy case is possible from FIG. 8, in which it is evident that NAF overfits the noise. FIG. 8 illustrates ground truth and reconstructions using all the methods applied to noisy projections. The top layer shows 50 projections, the bottom layer 400 projections, grayscale with density in [0-0.04]. CondCBNT does not overfit the noise and maintains tissue contrast. For an improved viewing experience, larger-scale versions of the experimental results are included in FIGS. 9 and 10, with FIG. 10 showing a volume with less noise in the projections.

FIG. 9 illustrates ground truth and reconstructions using all the methods applied to noisy projections. The top layer shows 50, the bottom layer 400 projections, grayscale colormap with density in [0-0.04]. The detector size causes a dense ring to appear in the FDK reconstruction. NAF overfits the noise with both 50 and 400 projections. Iterative over-smooths the soft tissues and removes bones. LIRE-L succeeds in keeping soft-tissue contrast and reconstructing bones. CondCBNT succeeds in not overfitting the noise and maintaining higher tissue contrast.

FIG. 10 illustrates ground truth and reconstructions using all the methods applied to noisy projections. The top layer shows 50, the bottom layer 400 projections, grayscale colormap with density in [0-0.04]. Similar to the FIG. 5, soft-tissue contrast resolution is very clear for CondCBNT and LIRE-L, thanks to less noise in the projections. NAF still overfits the noise. Less over-smoothing by the iterative method is observed.

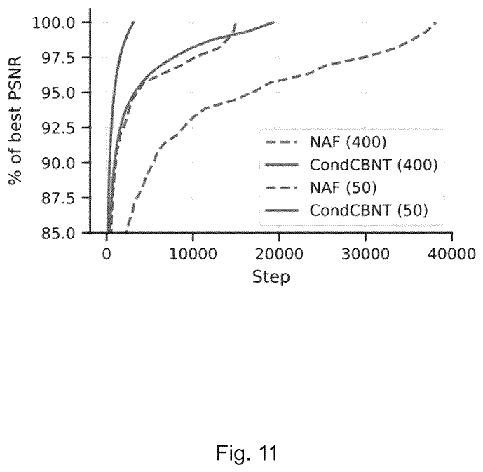

It can be seen from FIGS. 9 and 10 that in general, the iterative method over-smooths the reconstruction and exhibits blocky artifacts. The FDK reconstruction suffers from artifacts caused by the detector size, noise, and the low number of projections. LIRE-L and CondCBNT both reconstruct the volume with better soft-tissue contrast and without overfitting the noise. Comparing convergence speeds from Table 1 is difficult owing to diverging implementation choices and final performance reached. Consequently, performance was normalized by max mum PSNR reached after optimization. Additionally, given that dataset and batch size were the same, comparison was made using the number of iterations instead of wall-clock time. FIG. 11 illustrates the percentage of the best PSNR that a model can reach over the number of steps required to achieve it using noisy projections. CondCBNT converges significantly faster. This shows how CondCBNT quickly reaches a satisfying performance with both noisy and noise-free projections. It will be noted that, in the 400 projection case, CondCBNT was optimized for only half of a full epoch and still managed to outperform NAF and be within 1 standard deviation of LIRE-L. As the methods proposed in the present disclosure, of which CondCBNT is an implementation, do not require training the whole model from scratch for a newly obtained set of projections, the model converges considerably faster.

Example methods proposed according to the present disclosure therefore offer improved noise resistance of neural field (NF)-based CBCT reconstruction methods by sharing a conditional NF over scans taken from different patients. A continuous, local conditioning function is learned and expressed through a sample-(i.e., patient-) specific Neural Field, which modulates activations in the conditional NF to express volume-specific details, and may consequently be referred to as a Neural Modulation Field. In addition, examples of the present disclosure represent an efficient improvement over previous approaches, in terms of GPU memory scalability and reconstruction quality on both noise-free and noisy data and with varying numbers of available projections.

The methods of the present disclosure may be implemented in hardware, or as software modules running on one or more processors. The methods may also be carried out according to the instructions of a computer program, and the present disclosure also provides a computer readable medium having stored thereon a program for carrying out any of the methods described herein. A computer program embodying the disclosure may be stored on a computer readable medium, or it could, for example, be in the form of a signal such as a downloadable data signal provided from an Internet website, or it could be in any other form.

It should be noted that the above-mentioned examples illustrate rather than limit the disclosure, and that those skilled in the art will be able to design many alternative embodiments without departing from the scope of the appended claims or numbered embodiments. The word “comprising” does not exclude the presence of elements or steps other than those listed in a claim or embodiment, “a” or “an” does not exclude a plurality, and a single processor or other unit may fulfil the functions of several units recited in the claims or numbered embodiments. Any reference signs in the claims or numbered embodiments shall not be construed so as to limit their scope.

What is claimed is:

1. A computer implemented method for reconstructing a volumetric medical image of a patient from Cone Beam Computed Tomography (CBCT) projections of the patient, the computer implemented method comprising:
using a shared neural field to generate a volumetric field of attenuation coefficients from the CBCT projections, wherein the shared neural field is modulated by a patient specific neural field; and
mapping the volumetric field of attenuation coefficients to a volumetric image of the patient.

2. The computer implemented method of claim 1, wherein at least one of: 1) the shared neural field is operable to predict an attenuation coefficient value at a location within the volumetric field as a function of an input comprising three dimensional spatial coordinates of the location, 2) the shared neural field comprises an encoding layer operable to encode an input comprising three dimensional spatial coordinates into a multidimensional latent space, or 3) the encoding layer implements multiresolution hash encoding.

3. The computer implemented method of claim 1, wherein the shared neural field comprises a plurality of linear layers and a plurality of modulation layers, and wherein the patient specific neural field is operable to generate, as a function of an input comprising three dimensional spatial coordinates of a location, modulation vectors comprising parameters for the modulation layers of the shared neural field.

4. The computer implemented method of claim 3, wherein using the shared neural field to generate a volumetric field of attenuation coefficients from the CBCT projections comprises, for a given vector of 3D spatial coordinates input to the patient specific neural field, using the modulation vectors generated by the patient specific neural field to modulate the shared neural field when using the shared neural field to predict an attenuation coefficient at the location represented by the vector of 3D spatial coordinates input to the patient specific neural field.

5. The computer implemented method of claim 1, wherein using the shared neural field to generate a volumetric field of attenuation coefficients from the CBCT projections comprises:
training the patient specific neural field to represent specificities of a patient volume; and
using the shared neural field, modulated by the trained patient specific neural field, to generate the volumetric field of attenuation coefficients from the CBCT projections.

6. The computer implemented method of claim 5, wherein training the patient specific neural field to represent specificities of the patient volume comprises using as ground truth measured values of radiation intensity from the CBCT projections.

7. The computer implemented method of claim 5, wherein training the patient specific neural field to represent specificities of the patient volume comprises:
repeating, until a convergence condition is satisfied:
using the shared neural field, modulated by the patient specific neural field with current values of patient specific neural field trainable parameters, to generate a trial volumetric field of attenuation coefficients from the CBCT projections;
for each of a plurality of ray paths represented in the CBCT projections, each ray path originating in a CBCT source and terminating at a CBCT sensor:
comparing a radiation intensity predicted at the termination of the ray path by the trial volumetric field of attenuation coefficients, to a measured radiation intensity at the termination of the ray path extracted from a corresponding CBCT projection; and
updating values of the trainable parameters of the patient specific neural field to optimize a function of the comparisons.

8. The computer implemented method of claim 7, wherein the radiation intensity predicted at the termination of the ray path by the trial volumetric field of attenuation coefficients comprises an integral of the trial volumetric field of attenuation coefficients along the ray path.

9. The computer implemented method of claim 8, wherein the integral is approximated by a summation of points sampled from locations along the ray path.

10. The computer implemented method of claim 1, further comprising:
initiating values of the patient specific neural field to randomly generated initial values.

11. A computer implemented method for training a shared neural field for use in reconstructing a volumetric medical image of a patient from Cone Beam Computed Tomography (CBCT) projections of the patient, wherein the shared neural field is operable to generate a volumetric field of attenuation coefficients from the CBCT projections, and wherein the shared neural field is modulated by a patient specific neural field, the computer implemented method comprising:
training the shared neural field by using one or more ground truth volumetric medial images reconstructed from diagnostic CT projections of one or more individuals other than the patient for which the shared neural field will be used.

12. The computer implemented method of claim 11, wherein at least one of: 1) the shared neural field is operable to predict an attenuation coefficient value at a location within the volumetric field as a function of an input comprising three dimensional spatial coordinates of the location, 2) the shared neural field comprises an encoding layer operable to encode an input comprising three dimensional spatial coordinates into a multidimensional latent space, or 3) the encoding layer implements multiresolution hash encoding.

13. The computer implemented method of claim 11, wherein the shared neural field comprises a plurality of linear layers and a plurality of modulation layers, and wherein the patient specific neural field is operable to generate, as a function of an input comprising three dimensional spatial coordinates of a location, modulation vectors comprising parameters for the modulation layers of the shared neural field.

14. The computer implemented method of claim 13, wherein the shared neural field is operable to generate a volumetric field of attenuation coefficients from the CBCT projections by, for a given vector of 3D spatial coordinates input to the patient specific neural field, using the modulation vectors generated by the patient specific neural field to modulate the shared neural field when using the shared neural field to predict an attenuation coefficient at the location represented by the vector of 3D spatial coordinates input to the patient specific neural field.

15. The computer implemented method of claim 11 wherein training the shared neural field further comprises:
using a training data set, wherein for each of a plurality of training patients, the training data set comprises:
a volumetric field of attenuation coefficients reconstructed from diagnostic CT projections of the training patient; and
a plurality of simulated projections generated from the volumetric field of attenuation coefficients, each simulated projection comprising added noise.

16. The computer implemented method of claim 15, wherein training the shared neural field further comprises:
training the shared neural field using volumetric fields and simulated projections from a plurality of training patients, wherein, for individual training patients from the plurality of training patients, the shared neural field is modulated by the patient specific neural field; and
training each of a plurality of patient specific neural fields on a volumetric field and a plurality of simulated projections from a single training patient.

17. The computer implemented method of claim 15, wherein training the shared neural field, further comprises, for individual training patients from the plurality of training patients:
training the patient specific neural field to represent features of a training patient volume that are specific to the training patient; and
training the shared neural field to generate a volumetric field of attenuation coefficients from the simulated projections.

18. The computer implemented method of claim 15, wherein training the shared neural field further comprises, for individual training patients from the plurality of training patients, repeating, until a convergence condition is satisfied:
using the shared neural field with current values of shared neural field trainable parameters, modulated by the patient specific neural field for the training patient with current values of patient specific neural field trainable parameters, to generate a trial volumetric field of attenuation coefficients from the simulated projections for the training patient;
comparing the trial volumetric field of attenuation coefficients to the volumetric field of attenuation coefficients reconstructed from the diagnostic CT projections of the training patient; and
updating values of the trainable parameters of the shared neural field and the patient specific neural field to optimize a function of the comparison.

19. The computer implemented method of claim 18, wherein at least one of: 1) for individual training patients from the plurality of training patients, values of trainable parameters for the shared neural field are initiated to the values following training on a previous training patient, or 2) for individual training patients from the plurality of training patients, values of trainable parameters for the patient specific neural field are initiated to randomly generated initial values.

20. A radiotherapy treatment apparatus comprising at least one of:
1) a reconstruction node for reconstructing a volumetric medical image of a patient from Cone Beam Computed Tomography (CBCT) projections of the patient, the reconstruction node comprising processing circuitry configured to cause the reconstruction node to:
use a shared neural field, neural field, to generate a volumetric field of attenuation coefficients from the CBCT projections, wherein the shared neural field is modulated by a patient specific neural field; and
map the volumetric field of attenuation coefficients to a volumetric image of the patient;
2) a training node for training a shared neural field for use in reconstructing a volumetric medical image of a patient from Cone Beam Computed Tomography (CBCT) projections of the patient, wherein the shared neural field is operable to generate a volumetric field of attenuation coefficients from the CBCT projections, and wherein the shared neural field is modulated by a patient specific neural field. and wherein the training node comprising processing circuitry configured to cause the training node to:
train the shared neural field by using as ground truth volumetric medial images reconstructed from diagnostic CT projections of patients other than the patient for which the shared neural field will be used.
